(19) United States
(12) Patent Application Publication
(10) Pub. No.: US 2013/0023738 A1
(43) Pub. Date: Jan. 24, 2013

(54) MOBILE PHONE FOR HEALTH INSPECTION AND METHOD USING SAME

(71) Applicants: TING-SHE CHANG, Tu-Cheng (TW); TSUNG-JEN CHUANG, Tu-Cheng (TW); SHIH-FANG WONG, Tu-Cheng (TW)

(72) Inventors: TING-SHE CHANG, Tu-Cheng (TW); TSUNG-JEN CHUANG, Tu-Cheng (TW); SHIH-FANG WONG, Tu-Cheng (TW)

(73) Assignee: HON HAI PRECISION INDUSTRY CO., LTD., Tu-Cheng (TW)

(21) Appl. No.: 13/304,295

(22) Filed: Nov. 23, 2011

(30) Foreign Application Priority Data
Jul. 22, 2011 (CN) 201110206542.0

(51) Int. Cl. A61B 5/00

(52) U.S. Cl. 600/301

(57) ABSTRACT

A mobile phone includes a temperature detection system, a vibration detection system, memory, a processor, and a touch display. The temperature detection system detects a body temperature of an user. The vibration detection system detects a pulse rate of the user. The memory is configured for storing a predetermined temperature range and a predetermined pulse rate range. The processor receives and compares the body temperature of the user with the predetermined temperature range, receives and compares the pulse rate of the user with the predetermined pulse rate range to generate comparing results. The processor generates a warning message according to the comparing results. The touch display is connected to the processor for receiving and displaying the warning message.

### BACKGROUND

1. Technical Field

The present disclosure relates to mobile phones, and more particularly, to a mobile phone which is configured for inspecting health of a user.

2. Description of Related Art

Nowadays, various equipments are used to inspect the health of human beings to find diseases at their early stages, or to confirm a state of health, or to obtain advices in terms of what are cautious about in a daily life. However, these equipments are normally located in hospitals and operated by specialized persons. Because people would always need to go the hospital for a health checkup, these inspections are inconvenient for people to know their health whenever they want.

Therefore, a new inspection device is desired to overcome the above-described shortcomings.

### BRIEF DESCRIPTION OF THE DRAWINGS

The components in the drawings are not necessarily drawn to scale, the emphasis instead being placed upon clearly illustrating the principles of at least one embodiment. In the drawings, like reference numerals designate corresponding parts throughout the various views.

FIG. 1 is a schematic, block diagram of a mobile phone according to a first embodiment of the present disclosure.

FIG. 2 is flowing chart showing a method for inspecting health of a user according to a first embodiment of present disclosure, using the mobile phone of FIG. 1.

FIG. 3 is a flowing chart showing a method for inspecting health of a user according to a second embodiment of present disclosure, using the mobile phone of FIG. 1.

FIG. 4 is a schematic, block diagram of a mobile phone for inspecting health according to a second embodiment of the present disclosure.

### DETAILED DESCRIPTION

Reference will now be made to the drawings to describe various inventive embodiments of the present disclosure in detail, wherein like numerals refer to like units throughout.

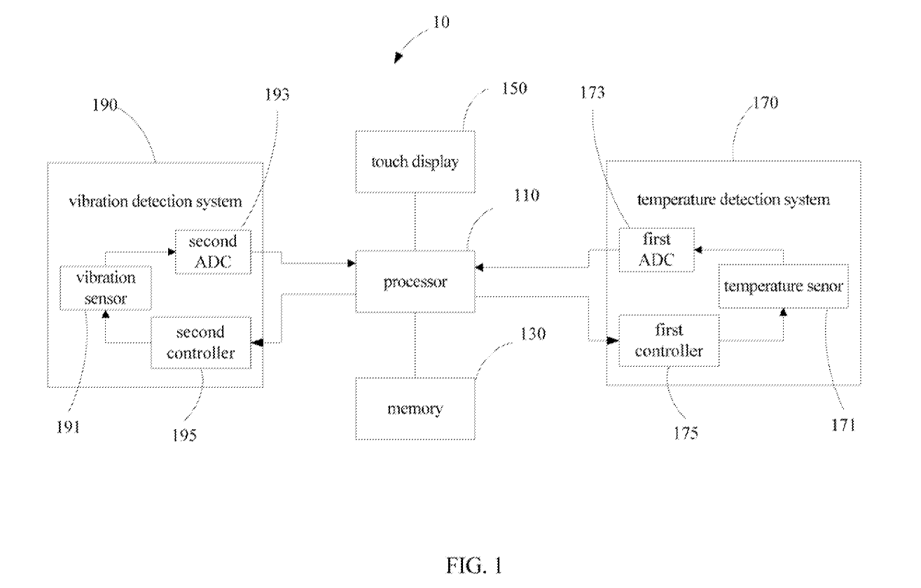

Referring to FIG. 1, a schematic, block diagram of a mobile phone according to a first embodiment of the present disclosure is shown. The mobile phone 10 includes a processor 110, a memory 130, a touch display 150, a temperature detection system 170, and a vibration detection system 190. The processor 110 is a multi-function processor and can perform a health inspection function or other functions such as communication, playing music, or creating office documents. The memory 130, the touch display 150, the temperature detection system 170, and the vibration detection system 190 are electronically connected to the processor 110.

The memory 130 is configured for storing a predetermined touch area parameter of the touch display 150, a predetermined temperature range, a predetermined pulse rate range, and emotion adjusting suggestions. In this embodiment, the predetermined temperature range includes a first temperature range of 35-36° C., a second temperature range of 36-38° C. and a third temperature range of 38-42° C. In this embodiment, the predetermined pulse rate range includes a first pulse rate range of 50-60 times/minute, a second pulse rate range of 60-100 times/minute, and a third pulse rate range of 100-120 times/minute. The second temperature range of 36-38° C. is defined as a normal temperature range. The first temperature range of 35-36° C. and the third temperature range of 38-42° C. are defined as an abnormal temperature range. The second pulse rate range of 60-100 times/minute is defined as a normal pulse rate range. The first pulse rate range of 50-60 times/minute and the third pulse rate range of 100-120 times/minute are defined as an abnormal pulse rate range.

When a user touch the touch display 150 with a finger or a touch pen, the touch display 150 is configured for sensing a touch action from the user, generating touch signals according to the touch action, and providing the touch signals to the processor 110. The processor 110 performs functions according to the touch signals. In one embodiment, the processor 110 analyzes a predetermined touch signal to obtain a current touch area of the touch display 150, then compares the current touch area with the predetermined touch area parameter from the memory 130. Under the condition that the current touch area is larger than the predetermined touch area parameter, the processor 110 performs a health inspection function and sends a temperature detecting command to the temperature detection system 170 and sends a vibration detecting command to the vibration detection system 190. In one embodiment, the predetermined touch area parameter can be one square centimeter and be calculated according known methods by one of ordinary skill in the art. In alternative embodiments, the processor 110 can automatically perform the health inspection function as soon as the mobile phone 10 is powered on. In this embodiment, the health inspection function can analyze certain health functions of a user, such as measuring heartbeat, measuring pulse rate, or measuring temperature.

The processor 110 controls operations of the vibration detection system 190 and the temperature detection system 170. The temperature detection system 170 is configured for detecting a body temperature of the user according to the temperature detecting command. The vibration detection system 190 is configured for detecting a pulse rate of the user according to the vibration detecting command.

In operation, the processor 110 first receives the body temperature from the temperature detection system 170 and receives the pulse rate from the vibration detection system 190. The processor 110 then reads the predetermined temperature range and the predetermined pulse rate from the memory. The processor 110 compares the body temperature of the user with the predetermined temperature range to obtain a first comparing result and compares the pulse rate of the user with the predetermined pulse rate range to obtain a second comparing result. The processor 110 determines a health situation of the user according to the first and the second comparing results, and generates corresponding warning messages according to the health situation of the user. The warning messages are received by touch display 150 and displayed on the touch display 150 for a predetermined time. In alternative embodiment, the touch display 150 can also display the body temperature of the user and the pulse rate of the user.

In one embodiment, when the body temperature of the user is in range of the first temperature range of 35-36° C. and the pulse rate of the user is in range of the second pulse rate range of 60-100 times/minute, the processor 110 generates a first warning message, such as congratulatory words of “thanks for maintaining your health”.

When the body temperature of the user is in range of the second temperature range of 35-36° C. and/or the pulse rate of the user is in range of the first pulse rate range of 50-60 times/minute or the third pulse rate range of 100-120 times/minute, the processor 110 generates a second warning message, such as suggestion words of “Breathe deeply now, please”, “Take it easy, please” or “Play some music, please”.

In one embodiment, when the processor 110 performs the health inspection function, the processor 110 simultaneously sends the temperature detecting command and the vibration detecting command to the temperature detection system 170 and the vibration detection system 190, correspondingly.

In alternative embodiment, the processor 110 first sends the temperature detecting command to the temperature detection system 170. The processor 110 only starts to send the vibration detecting command to the vibration detection system 190 when the body temperature of the user is in range of abnormal temperature range.

The temperature detection system 170 further includes a temperature sensor 171, a first analog to digital converter (ADC) 173, and a first controller 175. The first ADC 173 and the first controller 175 are connected to the processor 110. The temperature sensor 171 is connected between the ADC 173 and the first controller 175. In one embodiment, the temperature sensor 171 is integrated with a control component of the mobile phone 10, such as a power-on button, a backspace button, or a touch screen which is often touched by users.

When the first controller 175 receives the temperature detecting command from the processor 110, the first controller 175 sends a voltage signal to drive the temperature sensor 171. The temperature sensor 171 then starts to sense a body temperature of the user. The first ADC 173 transforms the body temperature into digital data and sends the digital data of the body temperature to the processor 110.

The vibration detection system 190 includes a vibration sensor 191, a second ADC 193, a second controller 195. The second ADC 193 and the second controller 195 are connected to the processor 110. The vibration sensor 191 is connected between the second ADC 193 and the second controller 195. In one embodiment, the vibration sensor 191 is also integrated with a control component of the mobile phone 10, such as a power-on button, a backspace button, or a touch screen which is often touched by users.

When the second controller 195 receives the vibration detecting command from the processor 110, the second controller 195 sends a voltage signal to drive the vibration sensor 191. The vibration sensor 191 then starts to sense a pulse rate of the user. The second ADC 193 transforms the pulse rate into digital data and sends the digital data of pulse rate to the processor 110.

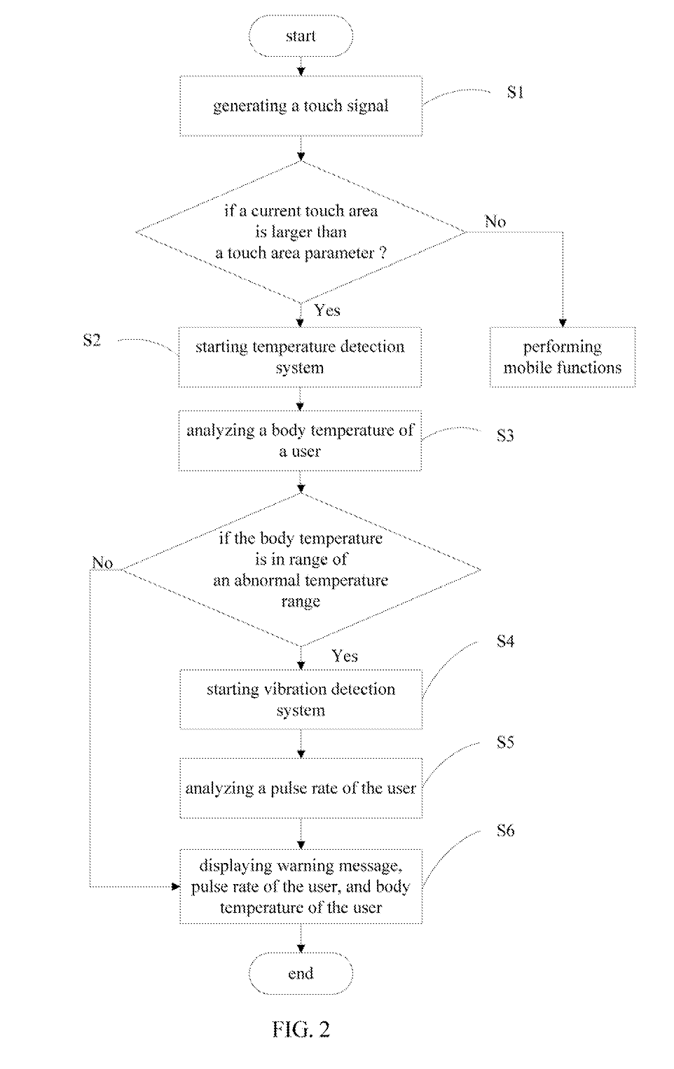

Referring to FIG. 2, a flowing chart showing a method for inspecting health of a user according to a first embodiment of present disclosure using the mobile phone 10 is shown. The method includes the following steps, but it should be understood that in other embodiments, additional steps may be added, others deleted, and the ordering of the steps may be changed.

In step S1, the touch display 150 receives a touch action from the user and generates the touch signal according to the touch action. The touch signal is then provided to the processor 110 from the touch display 150. The processor 110 simultaneously reads the predetermined touch area parameter from the memory 130 and analyzes the touch signal to obtain a current touch area of the touch display 150. After that, the processor 110 compares the current touch area with the predetermined touch area parameter to perform a health inspection function under the condition that the current touch area is larger than the predetermined touch area parameter under the condition that the current touch area is less than or equal to the predetermined touch area parameter, the processor 110 performs other functions.

In step S2, the processor 110 sends a temperature detecting command to the temperature detection system 170 as soon as the processor 110 perform the health inspection function. The temperature detection system 170 starts to work to obtain a body temperature of the user after receiving the detecting command from the processor 110.

In step S3, the processor 110 reads the predetermined temperature range from the memory 130 and receives the body temperature of the user from the temperature detection system 170. The processor 110 then compares if the body temperature of the user is in range of the abnormal temperature range of the predetermined temperature range. When the body temperature is in range of the abnormal temperature, step S4 is performed. When the body temperature is not in range of the abnormal temperature, step S6 is performed.

In step S4, the processor 110 sends a vibration detecting command to the vibration detection system 190. The vibration detection system 190 starts to work to obtain a pulse rate of the user after receiving the vibration detecting command from the processor 110.

In step S5, the processor 110 reads the predetermined pulse rate range from the memory 130 and receives the pulse rate of the user from the vibration detection system 190. The processor 110 then compares if the pulse rate of the user is in range of the abnormal pulse rate range of the predetermined pulse rate range.

In step S6, when the pulse rate of the user is not in range of the abnormal pulse rate range and the body temperature of the user is not in range of the abnormal temperature range, the touch display 150 displays a congratulatory words. When the pulse rate of the user is in range of the abnormal pulse rate range or/and the body temperature of the user is in range of the abnormal temperature range, the warning message such as “Breath deeply now, please”, “Take it easy, please” or “Playing some music, please” is displayed on the touch display 150. In this embodiment, the body temperature of the user and the pulse rate of the user are also displayed on the touch display 150. In alternative embodiment, the mobile phone can also play music when the pulse rate of the user is in range of the abnormal pulse rate range or/and the body temperature of the user is in range of the abnormal temperature range.

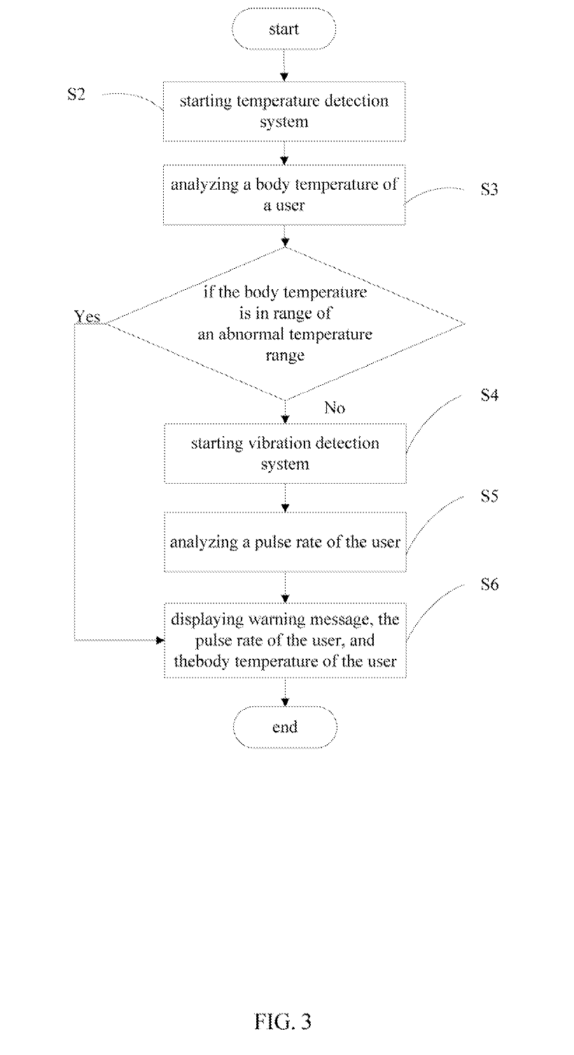

Referring to FIG. 3, a flowing chart showing a method for inspecting health of a user according to a second embodiment of present disclosure using the mobile phone 10 is shown. The method of second embodiment is similar to the first embodiment except that the first Step S1 is omitted and the processor 110 automatically performs the health inspection function to start the temperature detection system 170 as soon as the mobile phone is powered on.

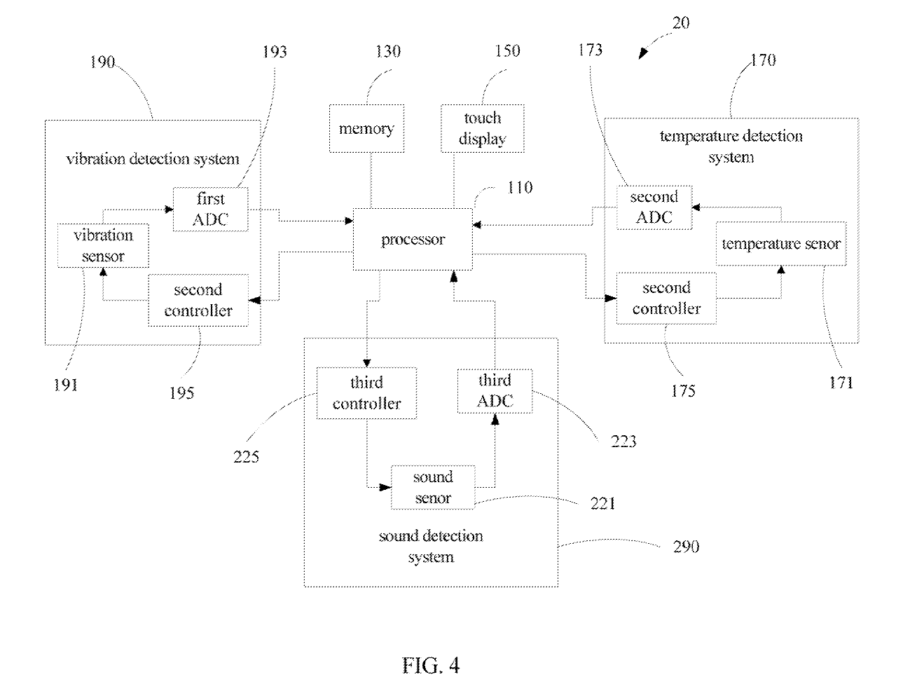

Referring to FIG. 4, a schematic, block diagram of a mobile phone for inspecting health according to a second embodiment is shown. An electrical device 20 is similar to the mobile phone 10 except that the electrical device 20 further includes a sound detection system 290. The sound detection system 290 includes a sound sensor 221, a third ADC 223, and a third controller 225. The third ADC 223 and the third controller 225 are connected to the processor 110. The sound sensor 221 is connected between the third ADC 223 and the third controller 225. In this embodiment, the sound sensor 221 may be a microphone.

When the processor 110 performs a health inspection function and the processor 110 sends a sound detecting command to the sound detection system 290. The third controller 225 sends a voltage signal to drive the sound sensor 221 after the third controller 225 receives the sound detecting command from the processor 110. The sound sensor 221 then starts to sense sound of the user. The third ADC 223 extracts a heartbeat from the sound of the user, and transforms the heartbeat into digital data and sends the heartbeat of the user to the processor 110.

The memory 130 further stores a predetermined heartbeat range. In this embodiment, the predetermined heartbeat range includes a first heartbeat range of 60-100 times/minute, a second heartbeat range of 50-60 times/minute, and a third heartbeat range of 100-120 times/minute. The first heartbeat range of 60-100 times/minute is defined as a normal heartbeat range. The second heartbeat range of 50-60 times/minute, and the third heartbeat range of 100-120 times/minute are defined as an abnormal heartbeat range.

The processor 110 receives the heartbeat of the user from the third ADC 223 and reads the predetermined heartbeat range from the memory 130, compares the heartbeat of the user with the predetermined heartbeat range to obtain a third comparing result, then determines a health situation of the user according to the three comparing results, and generates a corresponding warning message according to the health situation of the user. The warning message is finally received by the touch display 150 and displayed on the touch display 150 for a predetermined time. In this embodiment, the touch display 150 can also display the heartbeat of the user.

In one embodiment, when the heartbeat of the user is in range of the second heartbeat range of 60-100 times/minute, the processor 110 generates a first warning message, such as congratulatory words of “thanks for keeping your health”. When the heartbeat of the user is in range of the first heartbeat range of 50-60 times/minute or the third heartbeat range of 100-120 times/minute, the processor 110 generates a second warning message, such as suggestion words of “Breath deeply now, please”, “Take it easy, please” or “Playing some music, please”. In this embodiment, the touch display 150 can also display the heartbeat of the user.

In alternative embodiment, the warning message generated by the processor 110 can also be stored in the memory as the emotion adjusting suggestions. Historical data of the heartbeat, the body temperature, and the pulse rate of the user can also be stored in the memory.

It is to be understood, however, that even though numerous characteristics and advantages of certain inventive embodiments have been set out in the foregoing description, together with details of the structures and functions of the embodiments, the disclosure is illustrative only; and that changes may be made in detail, especially in matters of arrangement of parts within the principles of present invention to the full extent indicated by the broad general meaning of the terms in which the appended claims are expressed.

What is claimed is:

1. A mobile phone, comprising:
a temperature detection system configured for detecting a body temperature of a user;
a vibration detection system configured for detecting a pulse rate of the user;
a memory configured for storing a predetermined temperature range and a predetermined pulse rate range;
a processor configured for receiving and comparing the body temperature of the user with the predetermined temperature range, receiving and comparing the pulse rate of the user with the predetermined pulse rate range to generate comparing results, the processor configured for generating a warning message according to comparing results; and
a touch display connected to the processor for receiving and displaying the warning message.

2. The mobile phone of claim 1, wherein the processor automatically performs a health inspection function as soon as the mobile phone is powered on.

3. The mobile phone of claim 1, wherein the touch display is configured for receiving a touch action from a user and generating a touch signal according to the touch action.

4. The mobile phone of claim 3, wherein the memory is configured for storing a predetermined touch area parameter of the touch display, the processor analyzes the touch signal to obtain a current touch area of the touch display.

5. The mobile phone of claim 4, wherein the processor compares the current touch area with the predetermined touch area parameter to perform the health inspection function in response to the current touch area being larger than the predetermined touch area parameter.

6. The mobile phone of claim 5, wherein the processor sends a temperature detecting command to the temperature detection system and sends a vibration detecting command to the vibration detection system when the processor performs the health inspection function.

7. The mobile phone of claim 1, wherein the temperature detection system comprises a first analog to digital converter (ADC) connected to the processor, a first controller connected to the processor, and a temperature sensor connected between the first ADC and the first controller.

8. The mobile phone of claim 7, wherein the temperature sensor is integrated with an often used control component of the mobile phone.

9. The mobile phone of claim 1, wherein the predetermined temperature range comprises a first temperature range of 35-36° C., a second temperature range of 36-38° C. and a third temperature range of 38-42° C.

10. The mobile phone of claim 9, wherein the predetermined pulse rate range comprises a first pulse rate range of 50-60 times/minute, a second pulse rate range of 60-100 times/minute, and a third pulse rate range of 100-120 times/minute.

11. The mobile phone of claim 10, wherein the touch display displays the body temperature of the user and the pulse rate of the user.

12. The mobile phone of claim 10, wherein the processor generates a first warning message when the body temperature of the user is in range of the first temperature range of 35-36° C. and the pulse rate of the user is in range of the second pulse rate range of 60-100 times/minute.

13. The mobile phone of claim 12, wherein the first warning message comprises congratulatory words.

14. The mobile phone of claim 12, wherein the processor generates a second warning message when the body temperature of the user is in range of the second temperature range of 35-36° C. and/or the pulse rate of the user is in range of the first pulse rate range of 50-60 times/minute or the third pulse rate range of 100-120 times/minute.

15. The mobile phone of claim 14, wherein the second warning message comprises suggestion words.

16. The mobile phone of claim 1, further comprising a sound detection system configured for detecting heartbeat of the user.

17. The mobile phone of claim 1, wherein the processor generates a warning message when the heartbeat of the user is in range of 50-60 times/minute or 100-120 times/minute.

18. A method for inspecting health of a user using a mobile phone comprising a touch display, comprising:
analyzing a body temperature of a user by a temperature detection system of the mobile phone;
determining if the body temperature of the user is in range of an abnormal temperature range;
analyzing a pulse rate of the user by a vibration detection system of the mobile phone; and
determining if the pulse rate of the user is in range of an abnormal pulse rate range displaying a warning message on the touch display when the pulse rate of the user is in range of an abnormal pulse rate range, or the pulse rate of the user is in range of an abnormal pulse rate range.

19. The method for inspecting health of a user of claim 18, further comprising generating a touch signal by the touch display and determining if a current touch area corresponding to the touch signal is larger than a predetermined touch area parameter before analyzing the body temperature of the user.

20. The method for inspecting health of a user of claim 18, wherein the temperature detection system comprises a temperature sensor, and the vibration detection system comprises a vibration sensor.
